(19) United States
(12) Patent Application Publication
(10) Pub. No.: US 2009/0062622 A1
(43) Pub. Date: Mar. 5, 2009

(54) System and method for determining sleep status of a baby in a cradle and controlling movement of the cradle

(75) Inventor: Cing-Shin Lin, Tu-Cheng (TW)

Correspondence Address: PCE INDUSTRY, INC.;ATT. CHENG-JU CHIANG, 458 E. LAMBERT ROAD, FULLERTON, CA, 92835, US

(73) Assignees: PREMIER IMAGE Technology (China) LTD., Foshan City (CN); HON HAI Precision Industry CO., LTD., Tu-Cheng City (TW)

(21) Appl. No.: 11/985,732

(22) Filed: Nov. 16, 2007

(30) Foreign Application Priority Data
Aug. 31, 2007 (CN) 200710201571.1

(51) Int. Cl. A61B 5/00

(52) U.S. Cl. 600/300

(57) ABSTRACT

A computer-based method for determining sleep status of a baby in a cradle and controlling movement of the cradle is disclosed. The method includes the steps of: obtaining an image of the baby's face at fixed intervals through a digital camera, and storing the image in the digital camera; finding the baby's face in the image; locating the eyes in the image of the baby's face and framing the opening of the eyelids; determining the openness of the baby's eyes by calculating a ratio of a height of a rectangle framing the opening of eyelids to a width of the rectangle, and comparing the result with the preset threshold; judging whether the baby is asleep according to the comparison; sending a corresponding command to the motor for controlling the movement of the cradle. A related system is also disclosed.

### BACKGROUND OF THE INVENTION

1. Field of the Invention

The present invention is related to a system and method for determining sleep status of a baby in a cradle and controlling movement of the cradle (automatic system and method for helping a baby in a cradle go to sleep).

2. Description of Related Art

Usually, a baby sleeping in a cradle is monitored or checked by someone in the room with the baby. It is a hard job for a keeper especially during the night (rocking the cradle to help the baby go to sleep).

Therefore, what is needed is a system and method for determining sleep status of a baby in a cradle and controlling movement of the cradle, using a digital camera.

### SUMMARY OF THE INVENTION

A system for determining sleep status of a baby in a cradle and controlling movement of the cradle is provided in accordance with a preferred embodiment. The system includes a motor and a digital camera. The motor and the digital camera are installed on a beam of a stand of the cradle. The motor connects to the digital camera. The digital camera includes a sensor and a detecting module. The sensor is configured for obtaining an image of the baby's face. The detecting module includes a face-detecting sub-module, an eyes-detecting sub-module, and an analyzing sub-module. The face-detecting sub-module is configured for finding the baby's face in the image. The eyes-detecting sub-module is configured for locating the eyes in the image of the baby's face and framing the opening of the eyelids. The analyzing sub-module is configured for determining the openness of the baby's eyes by calculating a ratio of a height of a rectangle framing the opening of eyelids to a width of the rectangle, comparing the result with the preset threshold, judging whether the baby is asleep according to the comparison, and sending a corresponding command to the motor for controlling the movement of the cradle.

Other systems, methods, features, and advantages of the present invention will become apparent to one with skill in the art upon examination of the following drawings and detailed description.

### BRIEF DESCRIPTION OF THE DRAWINGS

FIG. 1 is a schematic diagram of hardware configuration of a system for determining sleep status of a baby in a cradle and controlling movement of the cradle in accordance with a preferred embodiment;

FIG. 2 is a schematic diagram showing function modules of the system of FIG. 1;

FIG. 3 is a flowchart of a preferred method for determining sleep status of a baby in a cradle and controlling movement of the cradle in accordance with one embodiment;

FIG. 4a to FIG. 4d are schematic diagrams showing the process of detecting a face of a baby;

FIG. 5a to FIG. 5f are schematic diagrams showing the process of detecting eyes of a baby; and

FIG. 6 is a schematic diagram showing a smallest rectangle framing an eyelid.

### DETAILED DESCRIPTION OF THE INVENTION

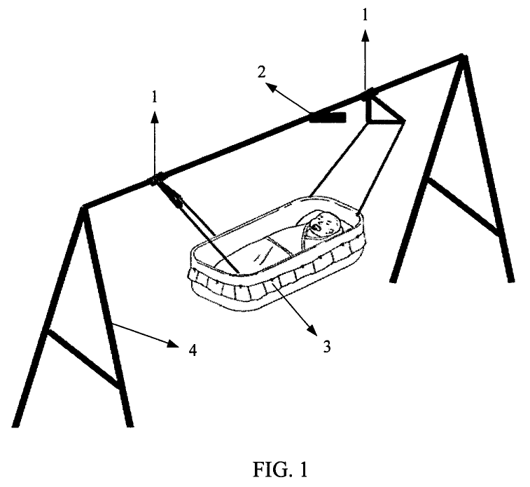

FIG. 1 is a schematic diagram of hardware configuration of a system for determining sleep status of a baby in a cradle and controlling movement of the cradle (hereinafter, “the system”) in accordance with a preferred embodiment. The system typically includes two motors 1, a digital camera 2, a cradle 3, and a stand 4. The two motors land the digital camera 2 are installed on a beam of the stand 4 of the cradle 3. The two motors 1 are connected to the ends of the cradle 3. The two motors 1 are configured for driving the cradle 3. A sensor of the digital camera 2 is aimed at the cradle 3. In order to obtain an image of the baby's face, the digital camera 2 is rotatably installed to follow the baby's face even while the cradle 3 is swinging.

The digital camera 2 is connected to the two motors 1 and is configured for obtaining an image of the baby's face, detecting a face and eyes in the image, determining the openness of the baby's eyes, judging whether the baby is asleep according to the result, and sending a corresponding command to the two motors 1 for controlling the movement of the cradle 3.

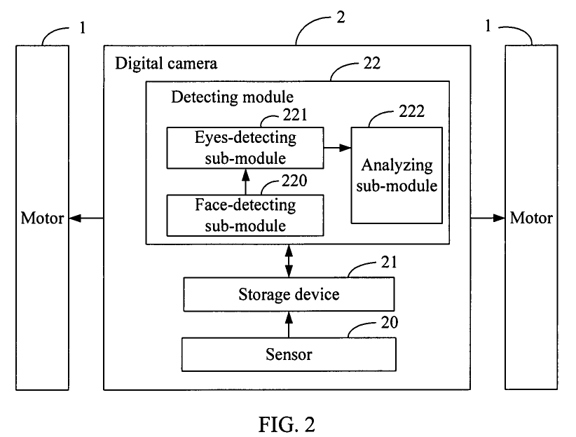

FIG. 2 is a schematic diagram showing function modules of the system of FIG. 1. The digital camera 2 may include a sensor 20, a storage device 21, and a detecting module 22. The detecting module 22 may include a face-detecting sub-module 220, an eyes-detecting sub-module 221, and an analyzing sub-module 222. The sensor 20 may be a CCD/CMOS sensor or any other suitable kind of sensor. The storage device 21 may be an SDRAM (Synchronous Dynamic Random Access Memory) device or any other suitable storage device.

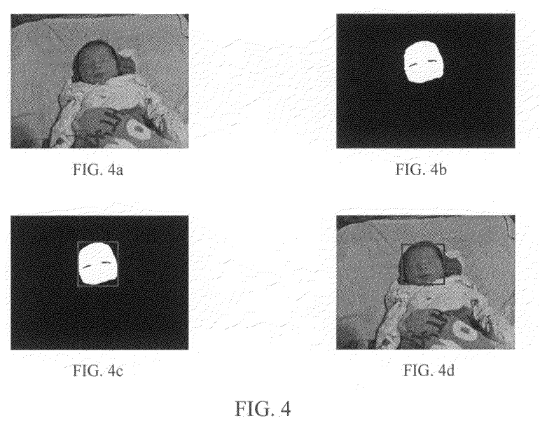

The sensor 20 is configured for obtaining an image of the baby's face (referring to FIG. 4a). The storage device 21 is configured for storing the image obtained by the sensor 20.

The face-detecting sub-module 220 is configured for finding the baby's face in the image. A detailed description is as follows. At first, the face-detecting sub-module 220 locates the baby's face (referring to FIG. 4b) by using an skin color model in YCbCr space. In YCbCr, the Y is the brightness (luma), Cb is blue minus luma (B−Y), and Cr is red minus luma (R−Y). An equation for judging facial skin is as follows.

$$P ( x , y ) = \{ \begin{matrix}1 , if [ 135 \leq Cr ( x , y ) \leq 156 ] ⋂ [ 108 \leq Cb ( x , y ) \leq 123 ] \\ 0 , otherwise\end{matrix}$$

In the equation, “P” stands for a pixel in the image, and “(x, y)” stands for a coordinate of “P”. In the preferred embodiment, a value of Cr(x, y) ranges from 135 to 156, a value of Cb(x, y) ranges from 108 to 123. In other embodiments, the values of the Cr(x, y) and the Cb(x, y) may be any other suitable ranges. If a value of the P(x, y) is 1, the face-detecting sub-module 220 judges an examined area in the image is facial skin. If a value of the P(x, y) is 0, the face-detecting sub-module 220 judges an examined area in the image is not facial skin. If it is facial skin, then that area of the image is set to white, otherwise, the area is set to black. Then, the face-detecting sub-module 220 calculates a smallest rectangle framing the face thereby identifying the baby's face in the image (referring to FIG. 4c and FIG. 4d).

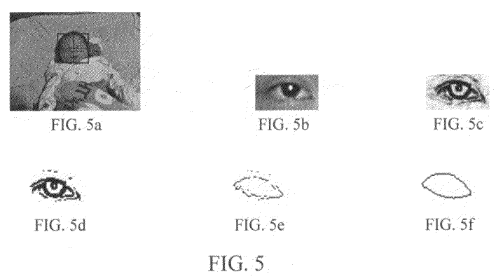

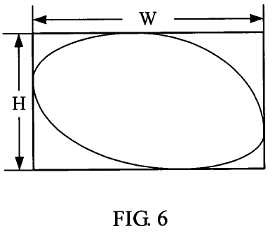

The eyes-detecting sub-module 221 is configured for locating the eyes in the image of the baby's face and framing the opening of the eyelids. A detailed description is as follows (the following description uses just one eye for example). At first, the eyes-detecting sub-module 221 locates an eye in the image of the baby's face (referring to FIG. 5a and FIG. 5b), and enhances the border of the eye image by using the Sobel algorithm thereby generating an enhanced eye image (referring to FIG. 5c). Then, the eyes-detecting sub-module 221 processes the enhanced eye image by using a binary image processing technique thereby generating a binary image of the eye (referring to FIG. 5d), samples points on the edge of the binary image of the eye (referring to FIG. 5e), and processes the sampled points by using the Snake algorithm thereby generating an outline of the opening of the eyelids of the eye (referring to FIG. 5f). At last, the eyes-detecting sub-module 221 calculates a smallest rectangle framing the outline of the opening of the eyelids (referring to FIG. 6). In FIG. 6, “H” stands for a height of the rectangle framing the outline of the opening of the eyelids, and “W” stands for a width of the rectangle framing the outline of the opening of the eyelids. In the preferred embodiment, the eye is shown open in the FIG. 5 and the FIG. 6.

The analyzing sub-module 222 is configured for determining the openness of the baby's eyes by calculating a ratio of “H” to “W”, comparing the result with the preset threshold, and judging whether the baby is asleep according to the comparison. If the ratio is greater than the preset threshold, the analyzing sub-module 222 judges the baby is not asleep. Otherwise, if the ratio is less than or equal to the preset threshold, the analyzing sub-module 222 judges the baby is asleep. Then, the analyzing sub-module 222 sends a corresponding command to the two motors 1 according to the result. The two motors 1 control the cradle 3 to swing or stop swinging according to the command. A detailed description is as follows. If the baby is asleep, the analyzing sub-module 222 sends a stop command to the two motors 1, and the two motors 1 stop running, whereupon the cradle 3 stops swinging. If the baby is not asleep, the analyzing sub-module 222 sends a run command to the two motors 1. The two motors 1 control a swing extent of the cradle 3 according to the run command.

In the preferred embodiment, the cradle is driven by the two motors 1, the digital camera 2 is rotatably fixed/installed on the beam of the stand 4 to follow the baby's face even while the cradle 3 is swinging. In other preferred embodiments, the cradle 3 can be driven by one motor.

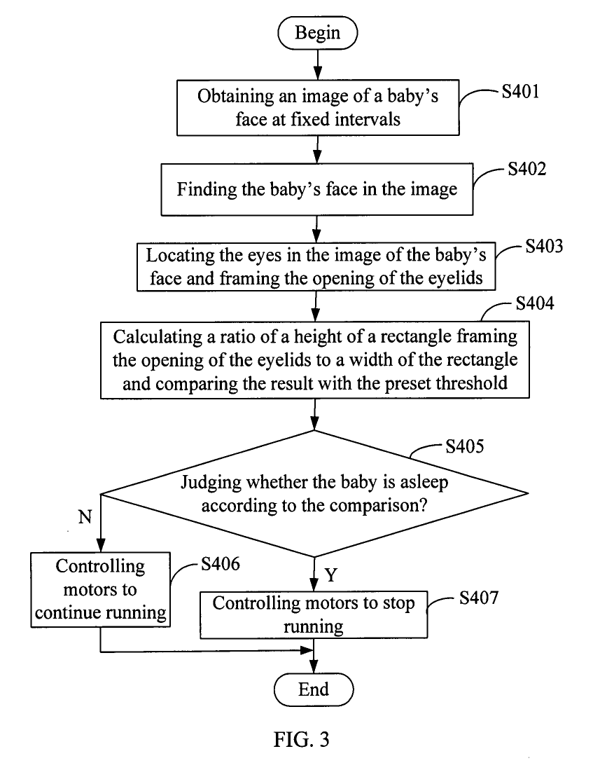

FIG. 3 is a flowchart of a preferred method for determining sleep status of a baby in a cradle and controlling movement of the cradle. In step S401, the sensor 20 in the digital camera 2 obtains an image of a baby's face (FIG. 4a) at fixed intervals, storing the images in the storage device 21.

In step S402, the face-detecting sub-module 220 finds the baby's face in a current image by using the method described in paragraphs 16 and 17.

In step S403, the eyes-detecting sub-module 221 locates the eyes in the image of the baby's face and frames the opening of the eyelids according to the methods described in paragraph 18.

In step S404, the analyzing sub-module 222 determines the openness of the baby's eyes by calculating a ratio of “H” to “W” and comparing the result with the preset threshold.

In step S405, the analyzing sub-module 222 judges whether the baby is asleep according to the comparison. If the ratio is greater than the preset threshold, the analyzing sub-module 222 judges the baby is not asleep. Otherwise, if the ratio is less than or equal to the preset threshold, the analyzing sub-module 222 judges the baby is asleep.

If the baby is not asleep, in step S406, the analyzing sub-module 222 sends a run command to the two motors 1. The two motors 1 control a swing extent of the cradle 3 according to the run command.

If the baby is asleep, in step S407, the analyzing sub-module 222 sends a stop command to the two motors 1, and the two motors 1 stop running, whereupon the cradle 3 stops swinging.

In the preferred embodiment, the cradle is driven by the two motors 1, the digital camera 2 is rotatably fixed/installed on the beam of the stand 4 to follow the baby's face even while the cradle 3 is swinging. In other preferred embodiments, the cradle 3 can be driven by one motor.

It should be emphasized that the above-described embodiments of the present invention, particularly, any preferred embodiments, are merely possible examples of implementations, merely set forth for a clear understanding of the principles of the invention. Many variations and modifications may be made to the above-described embodiment(s) of the invention without departing substantially from the spirit and principles of the invention. For example, the system can be used to judge whether the baby is crying according to a mouth shape of the baby or whether a quilt is covered on the baby through a face detecting, and notify a user through a warning voice or other suitable warning method. All such modifications and variations are intended to be included herein within the scope of this disclosure and the present invention and protected by the following claims.

What is claimed is:

1. A system for determining sleep status of a baby in a cradle and controlling movement of the cradle, the system comprising a motor and a digital camera, the motor and the digital camera being installed on a beam of a stand of the cradle, the motor being connected to the digital camera, the digital camera comprising a sensor and a detecting module, the sensor being configured for obtaining an image of the baby's face, the detecting module comprising:
a face-detecting sub-module configured for finding the baby's face in the image;
an eyes-detecting sub-module configured for locating the eyes in the image of the baby's face and framing the opening of the eyelids; and
an analyzing sub-module configured for determining the openness of the baby's eyes by calculating a ratio of a height of a rectangle framing the opening of eyelids to a width of the rectangle, comparing the result with the preset threshold, judging whether the baby is asleep according to the comparison, and sending a corresponding command to the motor for controlling the movement of the cradle.

2. The system according to claim 1, wherein the face-detecting sub-module finds the baby's face in the image by using an skin color model in YCbCr space.

3. The system according to claim 1, wherein the eyes-detecting sub-module finds the location of the eyes in the baby's face by using the Sobel algorithm and the Snake algorithm.

4. The system according to claim 1, wherein the compared result generated by the analyzing sub-module includes:
if the ratio is greater than the preset threshold, the analyzing sub-module judges the baby is not asleep; and
if the ratio is less than or equal to the preset threshold, the analyzing sub-module judges the baby is asleep.

5. The system according to claim 4, wherein the analyzing sub-module sends a corresponding command to the motor for controlling the movement of the cradle includes:
controlling the motor to stop running if the baby is asleep; and
controlling the motor to continue running if the baby is not asleep.

6. A method for determining sleep status of a baby in a cradle and controlling movement of the cradle, the method comprising:
obtaining an image of the baby's face at fixed intervals through a digital camera, and
storing the image in the digital camera, the digital camera being connected to a motor mounted on the cradle;
finding the baby's face in the image;
locating the eyes in the image of the baby's face and framing the opening of the eyelids;
determining the openness of the baby's eyes by calculating a ratio of a height of a rectangle framing the opening of eyelids to a width of the rectangle, and comparing the result with the preset threshold;
judging whether the baby is asleep according to the comparison; and
sending a corresponding command to the motor for controlling the movement of the cradle.

7. The method according to claim 6, wherein the baby's face in the image is found by using an skin color model in YCbCr space.

8. The method according to claim 6, wherein the location of the eyes in the baby's face is found by using the Sobel algorithm and the Snake algorithm.

9. The method according to claim 6, wherein the compared result includes:
if the ratio is greater than the preset threshold, judging the baby is not asleep; and
if the ratio is less than or equal to the preset threshold, judging the baby is asleep.

10. The method according to claim 9, wherein the step of sending a corresponding command to the motor for controlling the movement of the cradle includes:
controlling the motor to stop running if the baby is asleep; and
controlling the motor to continue running if the baby is not asleep.
